(19) United States
(12) Patent Application Publication
(10) Pub. No.: US 2010/0330090 A1
(43) Pub. Date: Dec. 30, 2010

(54) ANTAGONIST ANTIBODIES OF PROTEASE ACTIVATED RECEPTOR-1 (PAR1)

(75) Inventor: Kenneth R. Luehrsen, SAN DIEGO, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/669,467

(22) PCT Filed: Jul. 17, 2008
(86) PCT No.: PCT/US08/70357

§ 371 Date: Aug. 31, 2010

Related U.S. Application Data
(60) Provisional application No. 60/950,290, filed on Jul. 17, 2007.

(51) Int. Cl. A61K 39/395; C07K 16/28; A61P 35/00; A61P 9/10; A61P 1/00; A61P 19/04

(52) U.S. Cl. 424/135.1; 530/387.3; 530/389.6; 424/173.1

(57) ABSTRACT

The present invention provide antibodies or antigen-binding molecules that specifically recognize and antagonize the PAR1 receptor. Also provided in the invention are polynucleotides and vectors that encode such molecules and host cells that harbor the polynucleotides or vectors.

### CROSS-REFERENCE TO RELATED APPLICATIONS

This application claims the benefit of priority to U.S. Provisional Patent Application No. 60/950,290, filed 17 Jul. 2007. The full disclosure of this application is incorporated herein by reference in its entirety and for all purposes.

### BACKGROUND OF THE INVENTION

### Field of the Invention

The present invention relates to antibody and antigen binding molecule antagonists of protease activated receptor-1 (PAR1).

The protease-activated receptor 1 (PAR1) is a thrombin receptor which belongs to the class of G protein-coupled receptors (GCPR). PAR1 is expressed in various tissues, e.g., endothelial cells, smooth muscles cells, fibroblasts, neurons and human platelets. It is involved in cellular responses associated with hemostasis, proliferation, and tissue injury. Thrombin-mediated stimulation of platelet aggregation via PAR1 is an important step in clot formation and wound healing in blood vessels. Thrombin activates PAR1 by proteolytic removal of a portion of the extracellular N-terminal domain of PAR1 and exposing a new PAR1 N-terminus. The first few amino acids (SFLLRN; SEQ ID NO:68) of the new PAR1 N-terminus then act as a tethered ligand that binds to another part of the receptor to initiate signaling by an associated G-protein. PAR1 can also be activated by other serine proteases involved in blood clotting.

Modulation of PAR1-mediated signaling activities has several therapeutic applications. Inhibition of PAR1 is helpful for treating thrombotic and vascular proliferative disorders as well as for inhibiting progression of cancers. See, for example, Darmoul, et al., Mol Cancer Res (2004) 2(9):514-22 and Salah, et al, Mol Cancer Res (2007) 5(3):229-40. A PAR1 inhibitor, including an antagonist antibody or antigen binding molecule, has utility in the treatment of numerous disease conditions mediated by PAR1 intracellular signaling. For example, a PAR1 inhibitor, including an antagonist antibody or antigen binding molecule, finds use in preventing or inhibiting chronic intestinal inflammatory disorders, including inflammatory bowel disease (IBD), irritable bowel syndrome (IBS) and ulcerative colitis; and fibrotic disorders, including liver fibrosis and lung fibrosis. See, for example, Vergnolle, et al., J Clin Invest (2004) 114(10):1444; Yoshida, et al, Aliment Pharmacol Ther (2006) 24(Suppl 4):249; Mercer, et al., Ann NY Acad Sci (2007) 1096:86-88; Sokolova and Reiser, Pharmacol Ther (2007) PMID:17532472. A PAR1 inhibitor, including an antagonist antibody or antigen binding molecule, also finds use in preventing or inhibiting ischemia-reperfusion injury, including myocardial, renal, cerebral and intestinal ischemia-reperfusion injury. See, for example, Strande, et al., Basic Res. Cardiol (2007) 102(4):350-8; Sevastos, et al., Blood (2007) 109(2):577-583; Junge, et al., Proc Natl Acad Sci USA. (2003) 100(22):13019-24 and Tsuboi, et al., Am J Physiol Gastrointest Liver Physiol (2007) 292(2):G678-83 Inhibiting PAR1 intracellular signaling can also be used to inhibit herpes simple virus (HSV1 and HSV2) infection of cells. See, Sutherland, et al., J Thromb Haemost (2007) 5(5):1055-61.

### BRIEF SUMMARY OF THE INVENTION

The present invention provides improved antagonist antibodies against protease activated receptor-1 (PAR1) and methods for their use.

Accordingly, in one aspect, the invention provides antibodies that bind protease activated receptor-1 (PAR1). In some embodiments, the antibodies comprise:

(a) a heavy chain variable region comprising a human heavy chain V-segment, a heavy chain complementary determining region 3 (CDR3), and a heavy chain framework region 4 (FR4), and

(b) a light chain variable region comprising a human light chain V segment, a light chain CDR3, and a light chain FR4, wherein
i) the heavy chain CDR3 comprises the amino acid sequence DDX₁X₂SX₃WX₄FDV, wherein X₁ is G or I, X₂ is P or Y, X₃ is H, L, P, M, E, W, T, S, Q or A and X₄ is Y or F (SEQ ID NO:10);
ii) the light chain CDR3 variable region comprises the amino acid sequence FQGX₅X₆VPFT, wherein X₅ is S, D, A or V and X₆ is H, R, T, S or K (SEQ ID NO:20); wherein the antibody is a PAR1 antagonist.

In a related aspect, the invention provides antibodies that specifically bind PAR1. In some embodiments, the antibodies comprise a heavy chain variable region and a light chain variable region, wherein the heavy chain variable region and the light chain variable region each comprise the following three complementary determining regions (CDRs): CDR1, CDR2 and CDR3; wherein:

i) the CDR1 of the heavy chain variable region comprises an amino acid sequence selected from the group consisting of SEQ ID NO:3, SEQ ID NO:4 and SEQ ID NO:5;

ii) the CDR2 of the heavy chain variable region comprises an amino acid sequence selected from the group consisting of SEQ ID NO:7, SEQ ID NO:8 and SEQ ID NO:9;

iii) the CDR3 of the heavy chain variable region comprises an amino acid sequence selected from the group consisting of SEQ ID NO:11, SEQ ID NO:12 and SEQ ID NO:13;

iv) the CDR1 of the light chain variable region comprises an amino acid sequence selected from the group consisting of SEQ ID NO:15 and SEQ ID NO:16;

v) the CDR2 of the light chain variable region comprises an amino acid sequence selected from the group consisting of SEQ ID NO:18 and SEQ ID NO:19;

vi) the CDR3 of the light chain variable region comprises an amino acid sequence selected from the group consisting of SEQ ID NO:22 and SEQ ID NO:23. In some embodiments, the antibodies are a PAR1 antagonist.

In one embodiment, the heavy chain V-segment shares at least 90% sequence identity to SEQ ID NO:41, and the light chain V segment shares at least 90% sequence identity to SEQ ID NO:46.

In one embodiment, the heavy chain V-segment shares at least 90% sequence identity to an amino acid selected from the group consisting of SEQ ID NO:42, SEQ ID NO:43, SEQ ID NO:44 and SEQ ID NO:45, and the light chain V-segment shares at least 90% sequence identity to an amino acid selected from the group consisting of SEQ ID NO:47, SEQ ID NO:48, SEQ ID NO:49 and SEQ ID NO:50.

In one embodiment of the antibodies:

i) the heavy chain CDR3 comprises an amino acid sequence selected from the group consisting of SEQ ID NO:11, SEQ ID NO:12 and SEQ ID NO:13; and

ii) the light chain CDR3 comprises an amino acid sequence selected from the group consisting of SEQ ID NO:22 and SEQ ID NO:23.

In some embodiments, the heavy chain FR4 is a human germline FR4. In some embodiments, the heavy chain FR4 is human germline JH6 (WGQGTTVTVSS; SEQ ID NO:32). In some embodiments, the heavy chain J-segment comprises the human germline JH6 partial sequence DVWGQGTTVTVSS (SEQ ID NO:66).

In some embodiments, the light chain FR4 is a human germline FR4. In some embodiments, the light chain FR4 is human germline Jk2 (FGQGTKLEIK; SEQ ID NO:40). In some embodiments, the light chain J-segment comprises the human germline Jk2 partial sequence TFGQGTKLEIK (SEQ ID NO:67).

In some embodiments, the heavy chain V-segment and the light chain V-segment each comprise a complementary determining region 1 (CDR1) and a complementary determining region 2 (CDR2); wherein:

i) the CDR1 of the heavy chain V-segment comprises an amino acid sequence of SEQ ID NO:2;

ii) the CDR2 of the heavy chain V-segment comprises an amino acid sequence of SEQ ID NO:6;

iii) the CDR1 of the light chain V-segment comprises an amino acid sequence of SEQ ID NO:14; and

iv) the CDR2 of the light chain V-segment comprises an amino acid sequence of SEQ ID NO:17.

In some embodiments of the antibodies:

i) the CDR1 of the heavy chain V-segment comprises SEQ ID NO:4;

ii) the CDR2 of the heavy chain V-segment comprises SEQ ID NO:8;

iii) the heavy chain CDR3 comprises SEQ ID NO:11;

iv) the CDR1 of the light chain V-segment comprises SEQ ID NO:16;

v) the CDR2 of the light chain V-segment comprises SEQ ID NO:19; and

vi) the light chain CDR3 comprises SEQ ID NO:22.

In some embodiments of the antibodies:

i) the CDR1 of the heavy chain V-segment comprises SEQ ID NO:4;

ii) the CDR2 of the heavy chain V-segment comprises SEQ ID NO:8;

iii) the heavy chain CDR3 comprises SEQ ID NO:12;

iv) the CDR1 of the light chain V-segment comprises SEQ ID NO:16;

v) the CDR2 of the light chain V-segment comprises SEQ ID NO:19; and

vi) the light chain CDR3 comprises SEQ ID NO:23.

In some embodiments of the antibodies:

i) the CDR1 of the heavy chain V-segment comprises SEQ ID NO:5;

ii) the CDR2 of the heavy chain V-segment comprises SEQ ID NO:9;

iii) the heavy chain CDR3 comprises SEQ ID NO:13;

iv) the CDR1 of the light chain V-segment comprises SEQ ID NO:16;

v) the CDR2 of the light chain V-segment comprises SEQ ID NO:19; and

vi) the light chain CDR3 comprises SEQ ID NO:23.

In some embodiments, the heavy chain variable region shares at least 90% amino acid sequence identity to the variable region of SEQ ID NO:51 and the light chain variable region shares at least 90% amino acid sequence identity to the variable region of SEQ ID NO:55.

In some embodiments, the heavy chain variable region shares at least 90%, 93%, 95%, 96%, 97%, 98% or 99% amino acid sequence identity to the variable region selected from the group consisting of SEQ ID NO:52, SEQ ID NO:53 and SEQ ID NO:54 and the light chain variable region shares at least 90%, 93%, 95%, 96%, 97%, 98% or 99% amino acid sequence identity to the variable region selected from the group consisting of SEQ ID NO:57, SEQ ID NO:58 and SEQ ID NO:59.

In some embodiments, the heavy chain variable region comprises an amino acid sequence selected from the group consisting of SEQ ID NO:52, SEQ ID NO:53 and SEQ ID NO:54 and the light chain variable region comprises an amino acid sequence selected from the group consisting of SEQ ID NO:57, SEQ ID NO:58 and SEQ ID NO:59.

In some embodiments, the antibody binds to PAR1 with an equilibrium dissociation constant (KD) of less than 1×10⁻⁸ M.

In some embodiments, the antibody is a FAb′ fragment. In some embodiments, the antibody is an IgG. In some embodiments, the antibody is a single chain antibody (scFv). In some embodiments, the antibody comprises human constant regions.

In some embodiments, the antibody comprises a heavy chain comprising SEQ ID NO:52 and a light chain comprising SEQ ID NO:57.

In some embodiments, the antibody comprises a heavy chain comprising SEQ ID NO:53 and a light chain comprising SEQ ID NO:58.

In some embodiments, the antibody comprises a heavy chain comprising SEQ ID NO:54 and a light chain comprising SEQ ID NO:59.

In a further aspect, the invention provides pharmaceutically acceptable compositions comprising an antibody of the invention. The embodiments are as described herein.

In another aspect, the invention provides methods of ameliorating the symptoms of a disease condition mediated by intracellular signaling through PAR1 comprising administering to a subject in need thereof an antagonist antibody of the invention. The embodiments of the antibodies are as described herein.

In some embodiments of the methods, the disease condition mediated by aberrant intracellular signaling through PAR1 is a chronic intestinal inflammatory disorder.

In some embodiments of the methods, the disease condition mediated by aberrant intracellular signaling through PAR1 is a fibrotic disorder.

In some embodiments of the methods, the disease condition mediated by aberrant intracellular signaling through PAR1 is a cancer that overexpresses PAR1.

In some embodiments of the methods, the disease condition mediated by aberrant intracellular signaling through PAR1 is ischemia-reperfusion injury.

### DEFINITIONS

An “antibody” refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively. As used in this application, an “antibody” encompasses all variations of antibody and fragments thereof that possess a particular binding specifically, e.g., for PAR1. Thus, within the scope of this concept are full length antibodies, chimeric antibodies, single chain antibodies (ScFv), Fab, Fab′, and multimeric versions of these fragments (e.g., F(ab′)₂) with the same binding specificity.

“Complementarity-determining domains” or “complementary-determining regions (“CDRs”) interchangeably refer to the hypervariable regions of V_{L} and V_{H}. The CDRs are the target protein-binding site of the antibody chains that harbors specificity for such target protein. There are three CDRs (CDR1-3, numbered sequentially from the N-terminus) in each human V_{L} or V_{H}, constituting about 15-20% of the variable domains. The CDRs are structurally complementary to the epitope of the target protein and are thus directly responsible for the binding specificity. The remaining stretches of the V_{L} or V_{H}, the so-called framework regions, exhibit less variation in amino acid sequence (Kuby, Immunology, 4th ed., Chapter 4. W.H. Freeman & Co., New York, 2000).

The positions of the CDRs and framework regions are determined using various well known definitions in the art, e.g., Kabat, Chothia, international ImMunoGeneTics database (IMGT) (on the worldwide web at imgt.cines.fr/), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

The term “binding specificity determinant” or “BSD” interchangeably refer to the minimum contiguous or non-contiguous amino acid sequence within a complementary determining region necessary for determining the binding specificity of an antibody. A minimum binding specificity determinant can be within one or more CDR sequences. In some embodiments, the minimum binding specificity determinants reside within (i.e., are determined solely by) a portion or the full-length of the CDR3 sequences of the heavy and light chains of the antibody.

An “antibody light chain” or an “antibody heavy chain” as used herein refers to a polypeptide comprising the V_{L} or V_{H}, respectively. The endogenous V_{L} is encoded by the gene segments V (variable) and J (junctional), and the endogenous V_{H} by V, D (diversity), and J. Each of V_{L} or V_{H} includes the CDRs as well as the framework regions. In this application, antibody light chains and/or antibody heavy chains may, from time to time, be collectively referred to as “antibody chains.” These terms encompass antibody chains containing mutations that do not disrupt the basic structure of V_{L} or V_{H}, as one skilled in the art will readily recognize.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab′ which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. Paul, Fundamental Immunology 3d ed. (1993). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, (1992)).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some complementary determining region (“CDR”) residues and possibly some framework (“FR”) residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, and drug; or (b) the variable region, or a portion thereof, is altered, replaced or exchanged with a variable region having a different or altered antigen specificity.

The term “variable region” or “V-region” interchangeably refer to a heavy or light chain comprising FR1-CDR1-FR2-CDR2-FR3-CDR3-FR4. See, FIG. 1. An endogenous variable region is encoded by immunoglobulin heavy chain V-D-J genes or light chain V-J genes. A V-region can be naturally occurring, recombinant or synthetic.

As used herein, the term “variable segment” or “V-segment” interchangeably refer to a subsequence of the variable region including FR1-CDR1-FR2-CDR2-FR3. See, FIG. 1. An endogenous V-segment is encoded by an immunoglobulin V-gene. A V-segment can be naturally occurring, recombinant or synthetic.

As used herein, the term “J-segment” refers to a subsequence of the variable region encoded comprising a C-terminal portion of a CDR3 and the FR4. An endogenous J-segment is encoded by an immunoglobulin J-gene. see, FIG. 1. A J-segment can be naturally occurring, recombinant or synthetic.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other evaluated variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementary determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 92%, 94%, 96%, 98%, 99% sequence identity with the reference variable region nucleic acid or amino acid sequence. Corresponding human germline sequences can be determined, for example, through the publicly available international ImMunoGeneTics database (IMGT) (on the worldwide web at imgt.cines.fr/) and V-base (on the worldwide web at vbase.mrc-cpe.cam.ac.uk).

The phrase “specifically (or selectively) bind,” when used in the context of describing the interaction between an antigen, e.g., a protein, to an antibody or antibody-derived binding agent, refers to a binding reaction that is determinative of the presence of the antigen in a heterogeneous population of proteins and other biologics. Thus, under designated immunoassay conditions, the antibodies or binding agents with a particular binding specificity bind to a particular antigen at least two times the background and do not substantially bind in a significant amount to other antigens present in the sample. Specific binding to an antibody or binding agent under such conditions may require the antibody or agent to have been selected for its specificity for a particular protein. This selection may be achieved by subtracting out antibodies that cross-react with, e.g., PAR1 molecules from other species (e.g., mouse) or other PAR subtypes (e.g., PAR2, PAR3, PAR4). A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least than 10 to 100 times over the background.

The term “equilibrium dissociation constant (K_{D}, M)” refers to the dissociation rate constant (k_{d}, time⁻¹) divided by the association rate constant (kₐ, time⁻¹, M⁻¹). Equilibrium dissociation constants can be measured using any known method in the art. The antibodies of the present invention generally will have an equilibrium dissociation constant of less than about 10⁻⁸ M, for example, less than about 10⁻⁹ M or 10⁻¹⁰ M, in some embodiments, less than about 10⁻¹¹ M, 10⁻¹² M or 10⁻¹³ M.

As used herein, the term “antigen-binding region” refers to a domain of the PAR1-binding molecule of this invention that is responsible for the specific binding between the molecule and PAR1. An antigen-binding region includes at least one antibody heavy chain variable region and at least one antibody light chain variable region. There are at least one such antigen-binding regions present in each PAR1-binding molecule of this invention, and each of the antigen-binding regions may be identical or different from the others. In some embodiments, at least one of the antigen-binding regions of a PAR1-binding molecule of this invention acts as an antagonist of PAR1.

The term “antagonist,” as used herein, refers to an agent that is capable of specifically binding and inhibiting signaling through a receptor to fully block or detectably inhibit a response mediated by the receptor. For example, an antagonist of PAR1 specifically binds to the receptor and fully or partially inhibits PAR1-mediated signaling. In some cases, a PAR1 antagonist can be identified by its ability to bind to PAR1 and inhibit thrombin-induced calcium flux or thrombin-induced IL-8 production subsequent to intracellular signaling from a PAR1 (e.g., as measured in a FlipR assay, or by ELISA). Additional assays are described by Kawabata, et al., J Pharmacol Exp Ther. (1999) 288(1):358-70 Inhibition occurs when PAR1 intracellular signaling, as measured for example by calcium flux or IL-8 production, from a PAR1 exposed to an antagonist of the invention is at least about 10% less, for example, at least about 25%, 50%, 75% less, or totally inhibited, in comparison to intracellular signaling from a control PAR1 not exposed to an antagonist. A control PAR1 can be exposed to no antibody or antigen binding molecule, an antibody or antigen binding molecule that specifically binds to another antigen, or an anti-PAR1 antibody or antigen binding molecule known not to function as an antagonist. An “antibody antagonist” refers to the situation where the antagonist is an inhibiting antibody.

The term “protease activated receptor-1,” “proteinase activated receptor-1,” or “PAR1” interchangeably refer to a G-protein-coupled receptor that is activated by thrombin cleavage thereby exposing an N-terminal tethered ligand. PAR1 is also known as “thrombin receptor” and “coagulation factor II receptor precursor.” See, for example, Vu, et al., Cell (1991) 64(6):1057-68; Coughlin, et al, J Clin Invest (1992) 89(2):351-55; and GenBank Accession number NM_{—}001992. Intramolecular binding of the tethered ligand to the extracellular domain of PAR1 elicits intracellular signaling and calcium flux. See, for example, Traynelis and Trejo, Curr Opin Hematol (2007) 14(3):230-5; and Hollenberg, et al, Can J Physiol Pharmacol. (1997) 75(7):832-41. The nucleotide and amino acid sequences of PAR1 are known in the art. See, for example, Vu, et al., Cell (1991) 64(6):1057-68; Coughlin, et al, J Clin Invest (1992) 89(2):351-55; and GenBank Accession number NM_{—}001992. The nucleic acid sequence of human PAR1 is published as GenBank accession number NM_{—}001992 (see also, M62424.1 and gi4503636). The amino acid sequence of human PAR1 is published as NP_{—}001983 and AAA36743. As used herein, a PAR1 polypeptide is functionally a G-protein-coupled receptor that is activated by thrombin, and elicits intracellular signaling and calcium flux upon binding of the N-terminal tethered ligand. Structurally, a PAR1 amino acid sequence shares at least about 90%, 91%, 92%, 93%, 94%, 95%, 96%, 97%, 98%, 99% or 100% sequence identity with the amino acid of GenBank accession numbers NP_{—}001983, AAA36743 or M62424.1. Structurally, a PAR1 nucleotide acid sequence shares at least about 90%, 91%, 92%, 93%, 94%, 95%, 96%, 97%, 98%, 99% or 100% sequence identity with the amino acid of GenBank accession numbers NM_{—}001992, or M62424.1.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The invention provides polypeptides or polynucleotides that are substantially identical to the polypeptides or polynucleotides, respectively, exemplified herein (e.g., the CDRs exemplified in any one of SEQ ID NOS:2-23). Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “link,” when used in the context of describing how the antigen-binding regions are connected within a PAR1-binding molecule of this invention, encompasses all possible means for physically joining the regions. The multitude of antigen-binding regions are frequently joined by chemical bonds such as a covalent bond (e.g., a peptide bond or a disulfide bond) or a non-covalent bond, which can be either a direct bond (i.e., without a linker between two antigen-binding regions) or indirect bond (i.e., with the aid of at least one linker molecule between two or more antigen-binding regions).

The term “therapeutically acceptable amount” refers to an amount sufficient to effect the desired result (i.e., apoptosis of a target cell). Preferably, a therapeutically acceptable amount does not affect undesirable side effects. A therapeutically acceptable amount can be determined by first administering a low dose, and then incrementally increasing that dose until the desired effect is achieved.

### BRIEF DESCRIPTION OF THE DRAWINGS

FIG. 1 illustrates a schematic depiction of the structural units that comprise the V-regions of an antibody. The heavy chain is encoded by three gene families (Heavy-V, D and J) and the light chain is encoded by two gene families (Kappa or Lambda V and J). The recombination of these genes results in the intact V-region. The CDR3 sequences are at the recombination sites of the heavy V, D and J genes, in the case of the heavy chain, and the kappa or lambda V and J genes in the case of the light chain.

FIG. 2 illustrates a schematic of the replacement of reference antibody amino acid sequence with human amino acid sequence.

FIG. 3 illustrates an alignment of the improved variable region amino acid sequences of the invention (heavy chain V-region, SEQ ID NOS:52, 53 and 54; light chain V-region, SEQ ID NOS:57, 58 and 59) in comparison to a corresponding human germline variable region (e.g., Vh1-46 (SEQ ID NOS:42 and 32); VKII A3 (SEQ ID NO:56)). The complementary determining regions (CDRs) are underlined. Bolded residues indicate differences from human germline and italicized residues indicate alterations in CDR3.

FIG. 4 illustrates the high binding specificity of the improved antibodies of the invention in an ELISA assay. The improved anti-PAR1 antibodies bind to PAR1, but not to the closely related proteins human PAR2 or mouse PAR1 or mouse PAR2. The indicated antibody clone was added at a concentration of 1 μg/ml.

FIG. 5 illustrates that the improved antibodies are reactive with Cynomolgus PAR1. Sequences in the Cyno and human antibody binding epitopes are identical (SFLLRNPNDKYEPFWEDEEKNESGLTE; SEQ ID NO:1).

FIGS. 6A-C illustrate that the improved anti-PAR1 antibodies inhibit thrombin-mediated activation of PAR1 in a dose dependent manner

FIG. 7 illustrates a comparison of three improved antagonist PAR1 antibodies of the invention.

### DETAILED DESCRIPTION

### Generally

The antibodies of the present invention specifically bind to protease activated receptor-1 (PAR1). In doing so, the antibodies may block the binding of a native ligand (e.g., thrombin), act as an antagonist or act as an agonist. In some embodiments, the anti-PAR1 antibodies of the present invention act as antagonists of a PAR1 receptor. A PAR1 antibody antagonist is an antibody that specifically binds PAR1 and inhibits or decreases PAR1-mediated intracellular signaling. The anti-PAR1 antibodies optionally can be multimerized and used according to the methods of this invention. The anti-PAR1 antibodies can be a full-length tetrameric antibody (i.e., having two light chains and two heavy chains), a single chain antibody (e.g., a ScFv), or a molecule comprising antibody fragments that form one or more antigen-binding sites and confer PAR1-binding specificity, e.g., comprising heavy and light chain variable regions (for instance, Fab′ or other similar fragments).

Anti-PAR1 antibody fragments can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc. When present, the constant regions of the anti-PAR1 antibodies can be any type or subtype, as appropriate, and can be selected to be from the species of the subject to be treated by the present methods (e.g., human, non-human primate or other mammal, for example, agricultural mammal (e.g., equine, ovine, bovine, porcine, camelid), domestic mammal (e.g., canine, feline) or rodent (e.g., rat, mouse, hamster, rabbit).

Anti-PAR1 antibodies or antigen-binding molecules of the invention also include single domain antigen-binding units which have a camelid scaffold. Animals in the camelid family include camels, llamas, and alpacas. Camelids produce functional antibodies devoid of light chains. The heavy chain variable (VH) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies. Camelid scaffold-based anti-PAR1 molecules with binding specificities of the mouse anti-PAR1 antibodies exemplified herein can be produced using methods well known in the art, e.g., Dumoulin et al., Nature Struct. Biol. 11:500-515, 2002; Ghahroudi et al., FEBS Letters 414:521-526, 1997; and Bond et al., Mol Biol. 332:643-55, 2003.

a. Anti-PAR1 Antibody Variable Regions

The variable regions of the anti-PAR1 antibodies of the present invention are derived from a reference monoclonal antibody known to bind PAR1 with high affinity, and acts as an antagonist. The antibodies are improved or optimized by reducing the amino acid sequence segments corresponding to a non-human species (e.g., mouse) and increasing the amino acid sequence segments corresponding to human germline amino acid sequences. In this way, sequences that could potentially induce an immune response in a human host against the anti-PAR1 antibodies are reduced, minimized or eliminated. Methods for engineering human antibodies have been described. See, e.g., U.S. Patent Publication No. 2005/0255552 and U.S. Patent Publication No. 2006/0134098, the disclosures of both of which are hereby incorporated herein by reference in their entirety for all purposes.

The improved anti-PAR1 antibodies of the invention are engineered human antibodies with V-region sequences having substantial amino acid sequence identity to human germline V-region sequences while retaining the specificity and affinity of a reference antibody. See, U.S. Patent Publication No. 2005/0255552 and U.S. Patent Publication No. 2006/0134098. The process of improvement identifies minimal sequence information required to determine antigen-binding specificity from the variable region of a reference antibody, and transfers that information to a library of human partial V-region gene sequences to generate an epitope-focused library of human antibody V-regions. A microbial-based secretion system can be used to express members of the library as antibody Fab′ fragments and the library is screened for antigen-binding Fab′s, for example, using a colony-lift binding assay. See, e.g., U.S. Patent Publication No. 2007/0020685. Positive clones can be further characterized to identify those with the highest affinity. The resultant engineered human Fab′s retain the binding specificity of the parent, reference anti-PAR1 antibody, typically have equivalent or higher affinity for antigen in comparison to the parent antibody, and have V-regions with a high degree of sequence identity compared with human germ-line antibody V-regions.

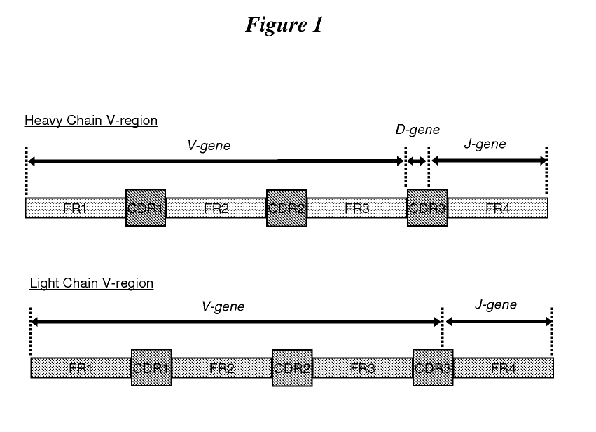

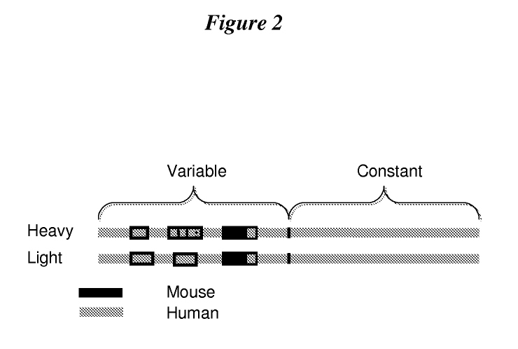

The minimum binding specificity determinant (BSD) required to generate the epitope-focused library is typically represented by a sequence within the heavy chain CDR3 (“CDRH3”) and a sequence within the light chain of CDR3 (“CDRL3”). The BSD can comprise a portion or the entire length of a CDR3. The BSD can be comprised of contiguous or non-contiguous amino acid residues. In some cases, the epitope-focused library is constructed from human V-segment sequences linked to the unique CDR3-FR4 region from the reference antibody containing the BSD and human germ-line J-segment sequences (see, FIG. 1 and U.S. Patent Publication No. 2005/0255552). Alternatively, the human V-segment libraries can be generated by sequential cassette replacement in which only part of the reference antibody V-segment is initially replaced by a library of human sequences. The identified human “cassettes” supporting binding in the context of residual reference antibody amino acid sequences are then recombined in a second library screen to generate completely human V-segments (see, U.S. Patent Publication No. 2006/0134098).

In each case, paired heavy and light chain CDR3 segments, CDR3-FR4 segments, or J-segments, containing specificity determinants from the reference antibody, are used to constrain the binding specificity so that antigen-binders obtained from the library retain the epitope-specificity of the reference antibody. Additional maturational changes can be introduced in the CDR3 regions of each chain during the library construction in order to identify antibodies with optimal binding kinetics. The resulting engineered human antibodies have V-segment sequences derived from the human germ-line libraries, retain the short BSD sequence from within the CDR3 regions and have human germ-line framework 4 (FR4) regions.

Accordingly, in some embodiments, the anti-PAR1 antibodies contain a minimum binding sequence determinant (BSD) within the CDR3 of the heavy and light chains derived from the originating monoclonal antibody. The remaining sequences of the heavy chain and light chain variable regions (CDR and FR), e.g., V-segment and J-segment, are from corresponding human germline amino acid sequences. The V-segments can be selected from a human V-segment library. Further sequence refinement can be accomplished by affinity maturation.

In another embodiment, the heavy and light chains of the anti-PAR1 antibodies contain a human V-segment from the corresponding human germline sequence (FR1-CDR1-FR2-CDR2-FR3), e.g., selected from a human V-segment library, and a CDR3-FR4 sequence segment from the originating monoclonal antibody. The CDR3-FR4 sequence segment can be further refined by replacing sequence segments with corresponding human germline sequences and/or by affinity maturation. For example, the FR4 and/or the CDR3 sequence surrounding the BSD can be replaced with the corresponding human germline sequence, while the BSD from the CDR3 of the originating monoclonal antibody is retained.

In some embodiments, the corresponding human germline sequence for the heavy chain V-segment is Vh1-46. In some embodiments, the corresponding human germline sequence for the heavy chain is J-segment is JH6. In some embodiments, the heavy chain J-segment comprises the human germline JH6 partial sequence DVWGQGTTVTVSS (SEQ ID NO:66). The full-length J-segment from human germline JH6 is YYYYYGMDVWGQGTTVTVSS. The variable region genes are referenced in accordance with the standard nomenclature for immunoglobulin variable region genes. Current immunoglobulin gene information is available through the worldwide web, for example, on the ImMunoGeneTics (IMGT), V-base and PubMed databases. See also, Lefranc, Exp Clin Immunogenet. 2001; 18(2):100-16; Lefranc, Exp Clin Immunogenet. 2001; 18(3):161-74; Exp Clin Immunogenet. 2001; 18(4):242-54; and Giudicelli, et al., Nucleic Acids Res. 2005 Jan. 1; 33(Database issue):D256-61.

In some embodiments, the corresponding human germline sequence for the light chain V-segment is VKII A3. In some embodiments, the corresponding human germline sequence for the light chain J-segment is Jk2. In some embodiments, the light chain J-segment comprises the human germline Jk2 partial sequence TFGQGTKLEIK. The full-length J-segment from human germline Jk2 is YTFGQGTKLEIK.

In some embodiments, the heavy chain V-segment shares at least 90%, 93%, 95%, 96%, 97%, 98%, 99% or 100% sequence identity to the amino acid sequence (Q/E)VQLVQSGAEVKKPG(A/S)SVKVSCK(A/V)SG(Y/G)TF(N/S)(N/S)Y(Y/V)(M/F) (N/H)WVRQAPGQGLEWMG(V/I)I(N/D)P(S/H)GG(R/S)T(R/S)Y(A/N)QKFKGRVTMT (T/R)DTSTST(V/A)YMELSSL(R/T)S(D/E)DTAVYYCAR (SEQ ID NO:41). In some embodiments, the light chain V-segment shares at least 90%, 93%, 95%, 96%, 97%, 98%, 99% or 100% sequence identity to the amino acid sequence

(SEQ ID NO: 46)
DIVMTQSPLSLPVTPGEPASISCRSSQSLLH(R/S)NG(Y/N)NYL
(D/E)WYLQKPGQSPQLLIY(K/L)(G/I)SNR(A/F)SGVPDRFS
GSGSGTDFTLKISRVEAEDVGVYYC.

In some embodiments, the heavy chain V-segment shares at least 90%, 93%, 95%, 96%, 97%, 98%, 99% or 100% sequence identity to an amino acid sequence selected from the group consisting of

(SEQ ID NO: 42)
QVQLVQSGAEVKKPGASVKVSCKASGYTFNSYYMHWVRQAPGQGLEWMGI
INPSGGSTSYAQKFQGRVTMTRDTSTSTVYMELSSLRSEDTAVYYCAR,
(SEQ ID NO: 43)
QVQLVQSGAEVKKPGASVKVSCKASGYTFNSYYMNWVRQAPGQGLEWMGV
IDPHGGRTRYNQKFKGRVTMTTDTSTSTVYMELSSLRSEDTAVYYCAR,
(SEQ ID NO: 44)
QVQLVQSGAEVKKPGASVKVSCKASGYTFNSYYMNWVRQAPGQGLEWMGV
IDPHGGRTRYNQKFKGRVTMTTDTSTSTAYMELRSLTSDDTAVYYCAR
and
(SEQ ID NO: 45)
EVQLVQSGAEVKKPGSSVKVSCKVSGGTFSNYVFNWVRQAPGQGLEWMGV
INPHSGRTRYNQKFKGRVTMTTDTSTSTAYMELRSLTSDDTAVYYCAR.

In some embodiments, the light chain V-segment shares at least 90%, 93%, 95%, 96%, 97%, 98%, 99% or 100% sequence identity to an amino acid sequence selected from the group consisting of

(SEQ ID NO: 47)
DIVMTQSPLSLPVTPGEPASISCRSSQSLLHSNGYNYLDWYLQKPGQSPQ
LLIYLGSNRASGVPDRFSGSGSGTDFTLKISRVEAEDVGVYYC,
(SEQ ID NO: 48)
DIVMTQSPLSLPVTPGEPASISCRSSQSLLHRNGNNYLEWYLQKPGQSPR
LLIYKISNRFSGVPDRFSGSGAGTDFTLKISRVEAEDVGVYYC,
(SEQ ID NO: 49)
DIVMTQSPLSLPVTPGEPASISCRSSQSLLHRNGNNYLEWYLQKPGQSPR
LLIYKISNRFSGVPDRFSGSGAGTDFTLKISRVEAEDVGVYYC
and
(SEQ ID NO: 50)
DIVMTQSPLSLPVTPGEPASISCRSSQSLLHRNGNNYLEWYLQKPGQSPR
LLIYKISNRFSGVPDRFSGSGAGTDFTLKISRVEAEDVGVYYC.

In some embodiments:
i) the heavy chain CDR3 comprises amino acid sequence motif DDX₁X₂SX₃WX₄FDV, wherein X₁ is G or I, X₂ is P or Y, X₃ is H, L, P, M, E, W, T, S, Q or A and X₄ is Y or F (SEQ ID NO:10),
ii) the light chain CDR3 comprises amino acid sequence motif FQGX₅X₆VPFT, wherein X₅ is S, D, A or V and X₆ is H, R, T, S or K (SEQ ID NO:20).

In some embodiments:
i) the heavy chain CDR3 comprises an amino acid sequence selected from the group consisting of DDGPSMWYFDV (SEQ ID NO:11), DDGPSLWYFDV (SEQ ID NO:12) and DDGPSHWYFDV (SEQ ID NO:13); and
ii) the light chain CDR3 comprises an amino acid sequence selected from the group consisting of MQALQTP (SEQ ID NO:21), FQGSSVPFT (SEQ ID NO:22) and FQGSHVPFT (SEQ ID NO:23).

In some embodiments, the antibodies of the invention comprise a heavy chain variable region comprising a CDR1 comprising an amino acid sequence S/N)Y(Y/V)(M/F)(N/H) (SEQ ID NO:2); a CDR2 comprising an amino acid sequence (I/V)I(N/D)P(S/H)(S/G)G(R/S)T(R/S)Y(A/N)QKF(K/Q)G (SEQ ID NO:6); and a CDR3 comprising an amino acid sequence of DDX₁X₂SX₃WX₄FDV, wherein X₁ is G or I, X₂ is P or Y, X₃ is H, L, P, M, E, W, T, S, Q or A and X₄ is Y or F (SEQ ID NO:10).

In some embodiments, the antibodies of the invention comprise a light chain variable region comprising a CDR1 comprising an amino acid sequence RSSQSLLH(R/S)NG(Y/N)NYL(D/E) (SEQ ID NO:14); a CDR2 comprising an amino acid sequence (L/K)(G/I)SNR(A/F)S (SEQ ID NO:17); and a CDR3 comprising an amino acid sequence of FQGX₅X₆VPFT, wherein X₅ is S, D, A or V and X₆ is H, R, T, S or K (SEQ ID NO:20).

In some embodiments, the heavy chain variable region comprises a FR1 comprising the amino acid sequence (E/Q)VQLVQSGAEVKKPG(S/A)SVKVSCK(V/A)SG(G/Y)TF(S/N) (SEQ ID NO:24); a FR2 comprising the amino acid sequence WVRQAPGQGLEWMG (SEQ ID NO:27); a FR3 comprising the amino acid sequence RVTMT(R/T)DTSTST(V/A)YMEL(S/R)SL(R/T)S(E/D)DTAVYYCAR (SEQ ID NO:28); and a FR4 comprising the amino acid sequence WGQGTTVTVSS (SEQ ID NO:32). The identified amino acid sequences may have one or more substituted amino acids (e.g., from affinity maturation) or one or two conservatively substituted amino acids.

In some embodiments, the light chain variable region comprises a FR1 comprising an amino acid sequence DIVMTQSPLSLPVTPGEPASISC (SEQ ID NO:33); a FR2 comprising the amino acid sequence WYLQKPGQSP(Q/R)LLIY (SEQ ID NO:34); a FR3 comprising the amino acid sequence GVPDRFSGSG(S/A)GTDFTLKISRVEAEDVGVYYC (SEQ ID NO:37); and a FR4 comprising the amino acid sequence FGQGTKLEIK (SEQ ID NO:40). The identified amino acid sequences may have one or more substituted amino acids (e.g., from affinity maturation) or one or two conservatively substituted amino acids.

Over their full length, the variable regions of the anti-PAR1 antibodies of the present invention generally will have an overall variable region (e.g., FR1-CDR1-FR2-CDR2-FR3-CDR3-FR4) amino acid sequence identity of at least about 90%, for example, at least about 91%, 92%, 93%, 94%, 95%, 96%, 97%, 98%, 99% or 100% to the corresponding human germline variable region amino acid sequence. For example, the heavy chain of the anti-PAR1 antibodies can share at least about 91%, 92%, 93%, 94%, 95%, 96%, 97%, 98%, 99% or 100% amino acid sequence identity to the human germline variable region Vh1-46/JH6. The light chain of the anti-PAR1 antibodies can share at least about 91%, 92%, 93%, 94%, 95%, 96%, 97%, 98%, 99% or 100% amino acid sequence identity to the human germline variable region VKII A3/Jk2.

In some embodiments, the anti-PAR1 antibodies of the invention comprise a heavy chain variable region having at least 90%, 91%, 92%, 93%, 94%, 95%, 96%, 97%, 98%, 99% or 100% amino acid sequence identity to a heavy chain variable region of SEQ ID NO:51 and comprise a light chain variable region having at least 90%, 91%, 92%, 93%, 94%, 95%, 96%, 97%, 98%, 99% or 100% amino acid sequence identity to a light chain variable region of SEQ ID NO:55.

In some embodiments, the anti-PAR1 antibodies of the invention comprise a heavy chain variable region having at least 90%, 91%, 92%, 93%, 94%, 95%, 96%, 97%, 98%, 99% or 100% amino acid sequence identity to a heavy chain variable region selected from the group consisting of SEQ ID NOS:52, 53 and 54 and comprise a light chain variable region having at least 90%, 91%, 92%, 93%, 94%, 95%, 96%, 97%, 98%, 99% or 100% amino acid sequence identity to a light chain variable region selected from the group consisting of SEQ ID NOS:56, 57, 58 and 59.

In some embodiments, the anti-PAR1 antibodies of the invention comprise a heavy chain variable region having at least 90%, 91%, 92%, 93%, 94%, 95%, 96%, 97%, 98%, 99% or 100% amino acid sequence identity to a heavy chain variable region of SEQ ID NO:52 and comprise a light chain variable region having at least 90%, 91%, 92%, 93%, 94%, 95%, 96%, 97%, 98%, 99% or 100% amino acid sequence identity to a light chain variable region of SEQ ID NO:57 (i.e., clone LDW653).

In some embodiments, the anti-PAR1 antibodies of the invention comprise a heavy chain variable region having at least 90%, 91%, 92%, 93%, 94%, 95%, 96%, 97%, 98%, 99% or 100% amino acid sequence identity to a heavy chain variable region of SEQ ID NO:53 and comprise a light chain variable region having at least 90%, 91%, 92%, 93%, 94%, 95%, 96%, 97%, 98%, 99% or 100% amino acid sequence identity to a light chain variable region of SEQ ID NO:58 (i.e., clone LDS 900).

In some embodiments, the anti-PAR1 antibodies of the invention comprise a heavy chain variable region having at least 90%, 91%, 92%, 93%, 94%, 95%, 96%, 97%, 98%, 99% or 100% amino acid sequence identity to a heavy chain variable region of SEQ ID NO:54 and comprise a light chain variable region having at least 90%, 91%, 92%, 93%, 94%, 95%, 96%, 97%, 98%, 99% or 100% amino acid sequence identity to a light chain variable region of SEQ ID NO:59 (i.e., clone LDS896).

For identified amino acid sequences less than 20 amino acids in length, one or two conservative amino acid residue substitutions can be tolerated while still retaining the desired specific binding and/or antagonist activity.

The anti-PAR1 antibodies of the present invention generally will bind PAR1 with an equilibrium dissociation constant (K_{D}) of less than about 10⁻⁸ M or 10⁻⁹ M, for example, less than about 10⁻¹⁰ M or 10⁻¹¹ M, in some embodiments less than about 10⁻¹² M or 10⁻¹³ M.

b. PAR1 Antibody Antagonists and Screening Methods

Any type of anti-PAR1 antibody antagonist may be used according to the methods of the present invention. Frequently, the antibodies used are monoclonal antibodies, which can be generated by any one of the methods known in the art (e.g., hybridomas and recombinant expression). In some embodiments, antibody fragments comprising heavy and light chain variable regions, rather than full-length antibodies, are used to construct the PAR1-binding molecule of this invention.

In some embodiments, the antigen-binding region(s) of the PAR1-binding molecule are single chain antibodies (ScFv). Techniques useful for producing ScFv and antibodies are known in the art, and described for example in, e.g., U.S. Pat. Nos. 4,946,778 and 5,258,498; Huston et al., Methods in Enzymology 203:46-88 (1991); Shu et al., Proc. Natl. Acad. Sci. USA 90:7995-7999 (1993); and Skerra et al., Science 240:1038-1040 (1988).

An anti-PAR1 antibody that specifically binds to PAR1 can be identified using techniques well known in the art, for example, ELISA, Surface Plasmon Resonance, interferometry (e.g., using ForteBio Octet biosensor system). A PAR1 antibody antagonist can be identified by testing for antibody's ability to inhibit or decrease PAR1-mediated events, e.g., calcium flux in a PAR1-expressing cell, inhibition of thrombin-induced IL-8 secretion, etc. A variety of assays known in the art can be used to detect induction of the presence and inhibition of PAR1-mediated events.

An PAR1-dependent calcium flux assay can be employed to screen for functional PAR1 antagonist antibodies. Thrombin is known to cleave the N-terminal domain of PAR1, removing approximately 15 amino acids. The remaining N-terminal domain, referred to as the tethered ligand, is responsible for the initiation of PAR1 mediated signaling. This cleavage of PAR-1 by thrombin results in a rapid and transient calcium flux in cells can be measured using commercially available reagents (e.g., commercially available from Molecular Devices). Specifically, antibodies can be evaluated using cells that express PAR1, e.g., HT-29, HCT-116 or DU145 cells, for their ability to inhibit calcium flux after thrombin treatment. Calcium flux can be measured using any method known in the art. In one example, cells in FlipR dye (Molecular Devices, Sunnyvale, Calif.) are pre-incubated with antibodies (e.g., about 1 hour), and Ca²⁺ flux is induced with thrombin at various concentrations. Antibodies can be purified using any method known in the art before testing for PAR1 antagonist activity. Control cells are not pre-incubated with antibody, or are pre-incubated with an antibody specific for an antigen other than PAR1, or are pre-incubated with an anti-PAR1 antibody known not to function as an antagonist. Thrombin-induced calcium flux is detectably inhibited or decreased in cells that are pre-incubated with a PAR1 antagonist antibody of the invention in comparison to thrombin-induced calcium flux in control cells. For example, thrombin-induced calcium flux will be decreased at least about 10%, for example, at least 30%, 50%, or 80%, or completely inhibited in cells exposed to the PAR1 antagonist antibody in comparison to control cells.

The PAR1 antagonist activity of the antibodies can also be determined by measuring inhibition of thrombin-mediated IL-8 secretion from appropriate target cells, for example HUVECs, by candidate anti-PAR1 antagonist antibodies. Thrombin-mediated IL-8 secretion from target cells can be measured using any method known in the art. In one example, cells are exposed to thrombin overnight which results in the elevation of IL-8 secreted into the media. In test samples, antibodies are added about 1 hour prior to thrombin treatment. IL-8 in the media can be measured by ELISA. Anti-PAR1 antagonist antibodies inhibit the increase in IL-8 secretion from target cells that were treated with thrombin, in comparison to control cells. Control cells are not pre-incubated with antibody, or are pre-incubated with an antibody specific for an antigen other than PAR1, or are pre-incubated with an anti-PAR1 antibody known not to function as an antagonist. Thrombin-induced IL-8 secretion is detectably inhibited or decreased in cells that are pre-incubated with a PAR1 antagonist antibody of the invention in comparison thrombin-induced IL-8 secretion from control cells. For example, thrombin-induced IL-8 secretion will be decreased at least about 10%, for example, at least 30%, 50%, or 80%, or completely inhibited in cells exposed to the PAR1 antagonist antibody in comparison to control cells.

c. Polynucleotides, Vectors and Host Cells for Producing Anti-PAR1 Antibodies

The invention provides polynucleotides (DNA or RNA) which encode polypeptides comprising segments or domains of the anti-PAR1 antibody chains or antigen-binding molecules described above. In some embodiments, the polynucleotides are substantially purified or isolated. Some of the polynucleotides of the invention comprise the polynucleotide sequence encoding a heavy chain variable region selected from the group consisting of SEQ ID NOS:51, 52, 53 and 54, and the polynucleotide sequence encoding a light chain variable region selected from the group consisting of SEQ ID NOS:55, 56, 57, 58 and 59. Some of the polynucleotides of the invention comprise the nucleotide sequence of the heavy chain variable region encoded by a polynucleotide sequence selected from the group consisting of SEQ ID NOS:60, 61 and 62, and the nucleotide sequence of the light chain variable region encoded by a polynucleotide sequence selected from the group consisting of SEQ ID NOS:63, 64 and 65. Some other polynucleotides of the invention comprise nucleotide sequences that are substantially identical (e.g., at least 70%, 80%, 95%, 96%, 97%, 98% or 99%) to one of the nucleotide sequences shown in SEQ ID NOS:60-65. When expressed from appropriate expression vectors, polypeptides encoded by these polynucleotides are capable of exhibiting antigen binding capacity.

Also provided in the invention are polynucleotides which encode at least one CDR region and usually all three CDR regions from the heavy or light chain of the mouse anti-PAR1 antibodies described in the Examples below. Some other polynucleotides encode all or substantially all of the variable region sequence of the heavy chain and/or the light chain of the mouse anti-PAR1 antibodies. For example, some of these polynucleotides encode the amino acid sequence having at least about 90%, 93%, 95%, 96%, 97%, 98%, 99% or 100% sequence identity to the heavy chain variable region shown in SEQ ID NOS:51, 52, 53 or 54 and/or the amino acid sequence having at least about 90%, 93%, 95%, 96%, 97%, 98%, 99% or 100% sequence identity to the light chain variable region shown in SEQ ID NOS:55, 56, 57, 58 and 59. Because of the degeneracy of the code, a variety of nucleic acid sequences will encode each of the immunoglobulin amino acid sequences.

The polynucleotides of the invention can encode only the variable region sequence of an anti-PAR1 antibody. They can also encode both a variable region and a constant region of the antibody. Some of polynucleotide sequences of the invention nucleic acids encode a mature heavy chain variable region sequence that is substantially identical (e.g., at least 80%, 90%, or 99%) to the mature heavy chain variable region sequence shown in SEQ ID NO:5 or 7. Some other polynucleotide sequences encode a mature light chain variable region sequence that is substantially identical to the mature light chain variable region sequence shown in SEQ ID NO:6 or 8. Some of the polynucleotide sequences encode a polypeptide that comprises variable regions of both the heavy chain and the light chain of one of the exemplified mouse anti-PAR1 antibody. Some other polynucleotides encode two polypeptide segments that respectively are substantially identical to the variable regions of the heavy chain and the light chain of one of the mouse antibodies.

The polynucleotide sequences can be produced by de novo solid-phase DNA synthesis or by PCR mutagenesis of an existing sequence (e.g., sequences as described in the Examples below) encoding an anti-PAR1 antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the anti-PAR1 antibodies described above. Various expression vectors can be employed to express the polynucleotides encoding the anti-PAR1 antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-PAR1 polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-PAR1 antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an anti-PAR1 antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-PAR1 antibody sequences. More often, the inserted anti-PAR1 antibody sequences are linked to a signal sequences before inclusion in the vector. Vectors to be used to receive sequences encoding anti-PAR1 antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the anti-PAR1 antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-PAR1 polypeptides of the invention. Insect cells in combination with baculovirus vectors can also be used.

In some preferred embodiments, mammalian host cells are used to express and produce the anti-PAR1 polypeptides of the present invention. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-PAR1 antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Properties of Anti-PAR1 Antibodies or Antigen-Binding Molecules

Once an anti-PAR1 antibody or antigen-binding molecule described above is synthesized or expressed from an expression vector in a host cell or endogenously in a hybridoma, they can be readily purified from, e.g., culture media and host cells. Usually, antibody chains are expressed with signal sequences and are thus released to the culture media. However, if antibody chains are not naturally secreted by host cells, the antibody chains can be released by treatment with mild detergent. Antibody chains can then be purified by conventional methods including ammonium sulfate precipitation, affinity chromatography to immobilized target, column chromatography, gel electrophoresis and the like. These methods are all well known and routinely practiced in the art, e.g., Scopes, Protein Purification, Springer-Verlag, NY, 1982; and Harlow & Lane, supra.

By way of example, selected hybridomas expressing anti-PAR1 antibodies of the invention can be grown in spinner-flasks for monoclonal antibody purification. Supernatants can be filtered and concentrated by affinity chromatography with protein A-sepharose or protein G-sepharose columns IgG molecules eluted from the columns can be examined by gel electrophoresis and high performance liquid chromatography to ensure purity. The buffer solution can be exchanged into PBS, and the concentration can be determined by OD280 reading. The monoclonal antibodies can be aliquoted and stored at −80° C.

Irrespective of their method of preparation, the anti-PAR1 antibodies or antigen-binding molecules of the present invention bind specifically to PAR1 or an antigenic fragment thereof. Specific binding exists when the dissociation constant for antibody binding to PAR1 or an antigenic fragment thereof is ≦1 μM, preferably ≦100 nM, and most preferably ≦1 nM. The ability of an antibody to bind to PAR1 can be detected by labeling the antibody of interest directly, or the antibody may be unlabelled and binding detected indirectly using various sandwich assay formats. See, e.g., Harlow & Lane, supra. Antibodies having such binding specificity are more likely to share the advantageous properties exhibited by the mouse or chimeric anti-PAR1 antibodies discussed in the Examples below. Typically, the anti-PAR1 antibodies or antigen-binding molecules of the invention bind to a PAR1 polypeptide or antigenic fragment with an equilibrium association constant (K_{A}) of at least 1×10⁷ M⁻¹, 10⁸ M⁻¹, 10⁹ M⁻¹, or 10¹⁰ M⁻¹. In addition, they also have a kinetic dissociation constant (k_{d}) of about 1×10⁻³ s⁻¹, 1×10⁻⁴ s⁻¹, 1×10⁻⁵ s⁻¹ or lower, and binds to human PAR1 (hPAR1) with an affinity that is at least two-fold greater than its affinity for binding to a non-specific antigen (e.g., BSA).

In some embodiments, the antibodies of the invention preferentially bind to human PAR1 over PAR1 from other mammalian species, for example, PAR1 from rodent species, including mouse, rat, rabbit and hamster. In some embodiments, the antibodies of the invention preferentially bind to PAR1 over other PAR subtypes, for example, PAR2, PAR3 or PAR4.

In some embodiments, the anti-PAR1 antibodies or antigen-binding molecules of the invention block or compete with binding of a reference anti-PAR1 antibody to an PAR1 polypeptide. The reference anti-PAR1 antibody can specifically bind to an epitope of PAR1 having the amino acid sequence comprising SFLLRNPNDKYEPFWEDEEKNESGLTE (SEQ ID NO:1), or fragments thereof, for example, a fragment of at least 8, 9, 10, 11, 12, 13, 14 or 15 contiguous amino acids. These can be fully human anti-PAR1 antibodies described above. They can also be other mouse, chimeric or humanized anti-PAR1 antibodies which bind to the same epitope as the reference antibody. The capacity to block or compete with the reference antibody binding to PAR1 indicates that an anti-PAR1 antibody or antigen-binding molecule under test binds to the same or similar epitope as that defined by the reference antibody, or to an epitope which is sufficiently proximal to the epitope bound by the reference anti-PAR1 antibody. Such antibodies are especially likely to share the advantageous properties identified for the reference antibody. The capacity to block or compete with the reference antibody may be determined by, e.g., a competition binding assay. With a competition binding assay, the antibody under test is examined for ability to inhibit specific binding of the reference antibody to a common antigen (e.g., a PAR1 polypeptide) or epitope on the antigen. A test antibody competes with the reference antibody for specific binding to the antigen or epitope if an excess of the test antibody substantially inhibits binding of the reference antibody. Substantial inhibition means that the test antibody reduces specific binding of the reference antibody usually by at least 10%, 25%, 50%, 75%, or 90%.

There are a number of known competition binding assays that can be used to assess competition of a test anti-PAR1 antibody or antigen-binding molecule with the reference anti-PAR1 antibody for binding to human PAR1 (hPAR1). These include, e.g., solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see Harlow & Lane, supra); solid phase direct label RIA using 1-125 label (see Morel et al., Molec. Immunol. 25:7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified antigen bound to a solid surface or cells bearing the antigen, an unlabelled test anti-PAR1 antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

To determine if a test anti-PAR1 antibody or antigen-binding molecule and a reference anti-PAR1 antibody bind to unique epitopes, each antibody can be biotinylated using commercially available reagents (e.g., reagents from Pierce, Rockford, Ill.). Competition studies using unlabeled monoclonal antibodies and biotinylated monoclonal antibodies can be performed using a PAR1 polypeptide coated-ELISA plates. Biotinylated MAb binding can be detected with a strep-avidin-alkaline phosphatase probe. To determine the isotype of a purified anti-PAR1 antibody, isotype ELISAs can be performed. For example, wells of microtiter plates can be coated with 1 μg/ml of anti-human IgG overnight at 4° C. After blocking with 1% BSA, the plates are reacted with 1 μg/ml or less of the monoclonal anti-PAR1 antibody or purified isotype controls, at ambient temperature for one to two hours. The wells can then be reacted with either human IgG1 or human IgM-specific alkaline phosphatase-conjugated probes. Plates are then developed and analyzed so that the isotype of the purified antibody can be determined.

To demonstrate binding of monoclonal anti-PAR1 antibodies or antigen-binding molecules to live cells expressing an PAR1 polypeptide, flow cytometry can be used. Briefly, cell lines expressing PAR1 (grown under standard growth conditions) can be mixed with various concentrations of an anti-PAR1 antibody in PBS containing 0.1% BSA and 10% fetal calf serum, and incubated at 37° C. for 1 hour. After washing, the cells are reacted with fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained as described above and examined by fluorescence microscopy.

Anti-PAR1 antibodies of the invention can be further tested for reactivity with an PAR1 polypeptide or antigenic fragment by immunoblotting. Briefly, purified PAR1 polypeptides or fusion proteins, or cell extracts from cells expressing PAR1 can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Therapeutic Applications and Pharmaceutical Compositions

The anti-hPAR1 antibodies or antigen-binding molecules described herein can be employed in many therapeutic or prophylactic applications by inhibiting PAR1 signaling activities. In therapeutic applications, a composition comprising an anti-hPAR1 antagonist antibody or antigen-binding molecule is administered to a subject already affected by a disease or condition mediated by, caused by or associated with PAR1 signaling. The composition contains the antibody or antigen-binding molecule in an amount sufficient to inhibit, partially arrest, or detectably slow the progression of the condition, and its complications.

In prophylactic applications, compositions containing the anti-hPAR1 antibodies or antigen-binding molecules are administered to a patient not already suffering from a PAR1-signaling related disorder. Rather, they are directed to a subject who is at the risk of, or has a predisposition, to developing such a disorder. Such applications allow the subject to enhance the patient's resistance or to retard the progression of a disorder mediated by PAR1 signaling.

Numerous disease conditions are mediated by aberrant or abnormally high PAR1-mediated intracellular signaling. Abnormally high PAR1-mediated intracellular signaling can be the caused by, for example, exposure of the receptor to abnormally high concentrations of an activating protease (e.g., thrombin) or abnormally high cell surface expression levels of PAR1. Inhibition of PAR1 is helpful for treating thrombotic and vascular proliferative disorders as well as for inhibiting progression of cancers, for example, carcinomas or epithelial cancers, including for example, skin cancers (including melanoma), gastrointestinal cancers (including colon cancer), lung cancer and mammary cancer (including breast and ductal cancers), prostate cancer, endometrial cancer, ovarian cancer, adenocarcinoma, and the like. See, for example, Darmoul, et al., Mol Cancer Res (2004) 2(9):514-22 and Salah, et al, Mol Cancer Res (2007) 5(3):229-40 Inhibiting PAR1 also finds use in preventing or inhibiting chronic intestinal inflammatory disorders, including inflammatory bowel disease (IBD), irritable bowel syndrome (IBS) and ulcerative colitis; and fibrotic disorders, including liver fibrosis and lung fibrosis. See, for example, Vergnolle, et al., J Clin Invest (2004) 114(10):1444; Yoshida, et al, Aliment Pharmacol Ther (2006) 24(Suppl 4):249; Mercer, et al., Ann NY Acad Sci (2007) 1096:86-88; Sokolova and Reiser, Pharmacol Ther (2007) PMID:17532472.

Cancers that can be inhibited or prevented by the anti-PAR1 antibodies of the invention include, without limitation, epithelial cancers including carcinomas; gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 1997) for additional cancers).

Inhibiting PAR1 also finds use in preventing or inhibiting disease conditions that may or may not be mediated by aberrant or abnormally high PAR1 expression or intracellular signaling. For example, inhibiting PAR1 is useful for preventing or inhibiting ischemia-reperfusion injury, including myocardial, renal, cerebral and intestinal ischemia-reperfusion injury. See, for example, Strande, et al., Basic Res. Cardiol (2007) 102(4):350-8; Sevastos, et al., Blood (2007) 109(2):577-583; Junge, et al., Proc Natl Acad Sci USA. (2003) 100(22):13019-24 and Tsuboi, et al., Am J Physiol Gastrointest Liver Physiol (2007) 292(2):G678-83. Inhibiting PAR1 intracellular signaling can also be used to inhibit herpes simple virus (HSV1 and HSV2) infection of cells. See, Sutherland, et al., J Thromb Haemost (2007) 5(5):1055-61.

The invention provides pharmaceutical compositions comprising the anti-hPAR1 antibodies or antigen-binding molecules formulated together with a pharmaceutically acceptable carrier. The compositions can additionally contain other therapeutic agents that are suitable for treating or preventing a given disorder. Pharmaceutically carriers enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the anti-hPAR1 antibody is employed in the pharmaceutical compositions of the invention. The anti-hPAR1 antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the antibodies of the invention employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

In embodiments where the agent is a nucleic acid, typical dosages can range from about 0.1 mg/kg body weight up to and including about 100 mg/kg body weight, preferably between about 1 mg/kg body weight to about 50 mg/kg body weight. More preferably, about 1, 2, 3, 4, 5, 10, 15, 20, 30, 40 or 50 mg/kg body weight.

Antibody is usually administered on multiple occasions. Intervals between single dosages can be weekly, monthly or yearly. Intervals can also be irregular as indicated by measuring blood levels of anti-hPAR1 antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The following examples are offered to illustrate, but not to limit the claimed invention.

### Example 1

The following example provides the construction and screening of anti-Par-1 antibodies with minimized immunogenicity in humans.

### Methods

### Sub-Cloning of Murine V-Regions

The murine V-regions were sub-cloned from the murine monoclonal 4E7.J14. PCR was used to amplify the V-genes of the V-heavy and V-Kappa regions and incorporate restriction enzyme sites suitable for cloning into expression vectors. V-regions were cloned as Fab′ fragments with human IgG1 constant regions in a pBR322 vector system. Fab′s were expressed from pTAC promoters in E. coli. The purified Fab′ protein (Clone PR15-5) was shown to bind Par-1 antigen in an ELISA assay.

### Fab′ and Fab Purification

Fab′ and Fab fragments were expressed by secretion from E. coli using expression vectors. Cells were grown in 2×YT medium to an optical density measured at 600 nm wavelength (OD₆₀₀) of 0.6. Expression was induced using isopropyl-beta-D-thiogalactopyranoside (IPTG) for 3 hours at 33° C. Assembled Fab′ or Fab was obtained from periplasmic fractions and purified by affinity chromatography using Streptococcal Protein G (HiTrap Protein G HP columns; GE Healthcare) according to standard methods. Fab′s and Fabs were eluted in pH 2.0 buffer, immediately adjusted to pH 7.0 and dialyzed against PBS pH7.4 (PBS is without calcium and magnesium).

### ELISA

Typically, 50 ng/well of Par-1 antigen was bound to a 96 well microtiter plate by overnight incubation at 4° C. The plate was blocked with a solution of 5% milk in phosphate-buffered saline containing 0.1% Tween20 (“PBST”) for one hour at 33° C. The Fabs or the reference Fab′ (PR15-5) were diluted in PBS and 50 μl was added to each well. After one hour incubation at 33° C., the plate was rinsed three times with PBST. 50 μl of anti-human-kappa chain horseradish peroxidase (HRP) conjugate (Sigma; diluted to 0.1 ng/ml in PBST) was added to each well and the plate was incubated for 40 min at 33° C. The plate was washed three times with PBST and once with PBS. 100 μl of 3,3′,5,5′-tetramethylbenzidine (TMB) substrate (Sigma) was added to each well and the plate was incubated for ˜5 min at room temperature. To stop the reaction, 100 μl of 0.2 N H₂SO₄ was added to each well. The plate was read at 450 nm in a spectrophotometer.

### Screening

Screening of libraries of Fab fragments was carried out as described in U.S. Patent Publication Nos. 2005/0255552 and 2006/0134098 using recombinant Par-1 antigen-coated nitrocellulose filters. The disclosure of both of these patent publications is hereby incorporated herein by reference in its entirety for all purposes.

### Affinity Measurements

The binding kinetics of the Fab′ and Fab fragments were analyzed using a ForteBio Octet biosensor. Recombinant antigen was biotinylated using the EZ-link biotinylation kit (Pierce) according to the manufacturer's methods. The antigen was then coupled to neutravidin-coated sensors (ForteBio). Fab′ and Fab binding was monitored in real time using bio-layer interferometry analysis and software provided by the manufacturer. Affinities were calculated from the determined association and dissociation constants.

### Results

### Cloning and Expression of V-Regions

### ForteBio Octet Test Confirming Par-1 Antigen Binding Activity for the Sub-Cloned V-Regions.

The murine V-regions were cloned, sequenced and expressed. V-regions were cloned as Fab′ fragments with human IgG1 constant regions and expressed in E. coli. In a ForteBio Octet test of Par-1 antigen binding, the cloned Fab′ PR15-5 produced a binding curve that was dependent on antibody concentration. See, Table 1.

TABLE 1

| | PR15-5 | PR15-5 |
|---|---|---|
| MolarConc [M] | 5E−8 | 2.5E−8 |
| kdis [1/s] | 2.43E−4 | 2.61E−4 |
| kassoc [1/Ms] | 2.97E5 | 2.97E5 |
| K_{D} [M] | 8.19E−10 | 8.77E−10 |

### Library Construction and V-Region Cassettes

Epitope-focused libraries were constructed from libraries of human V-segment sequences linked to the unique CDR3 regions of reference Fab′ PR15-5; the FR4 regions were human germ-line JH6 and Jk2 for the heavy and light chains, respectively. These “full-length” libraries were used as a base for construction of “cassette” libraries in which only part of the murine V-segment is initially replaced by a library of human sequences. The cassettes for both V-heavy and V-kappa chains were made by bridge PCR with overlapping common sequences within the framework 2 (FR2) region. In this way “front-end” and “middle” human cassette libraries were constructed for human V-heavy 1 and V-kappa II subclasses. Human cassettes which supported binding to Par-1-antigen were identified by colony-lift binding assay and ranked according to affinity in ELISA and ForteBio Octet biosensor analysis. Pools of the highest affinity cassettes were then recombined in a second library screen to generate completely human V-segments. Cassette screening completed in this way identified multiple human V-light chains and human V-heavy “front-end” cassettes that had Par-1 antigen binding activity.

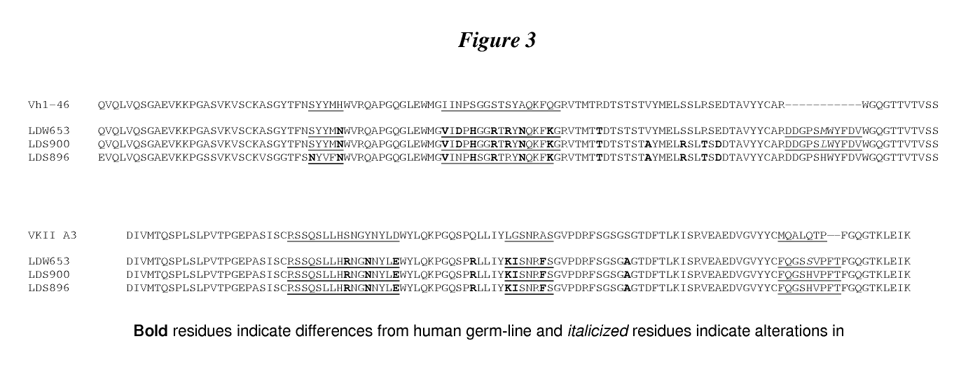

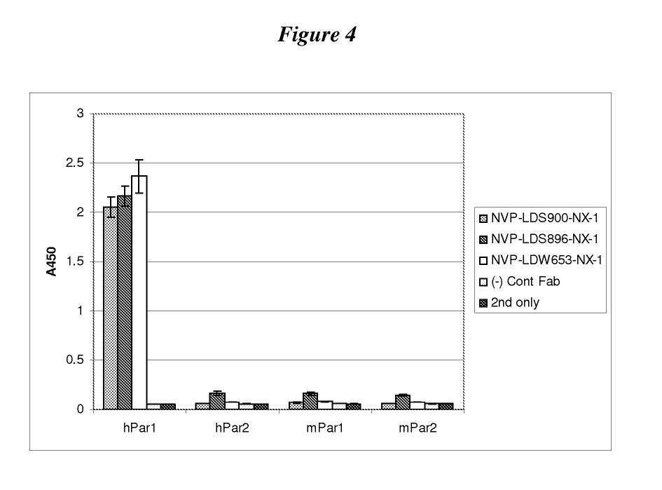

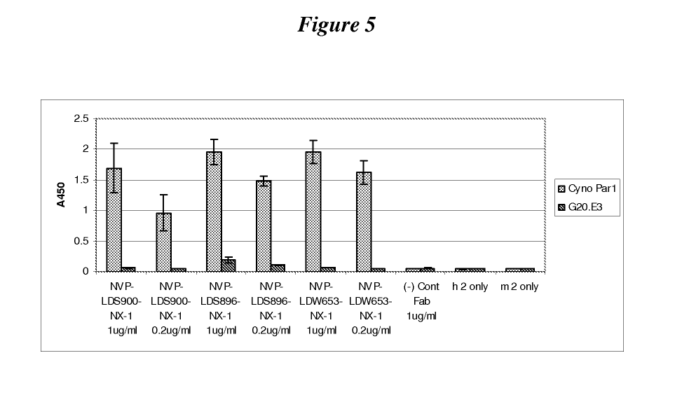

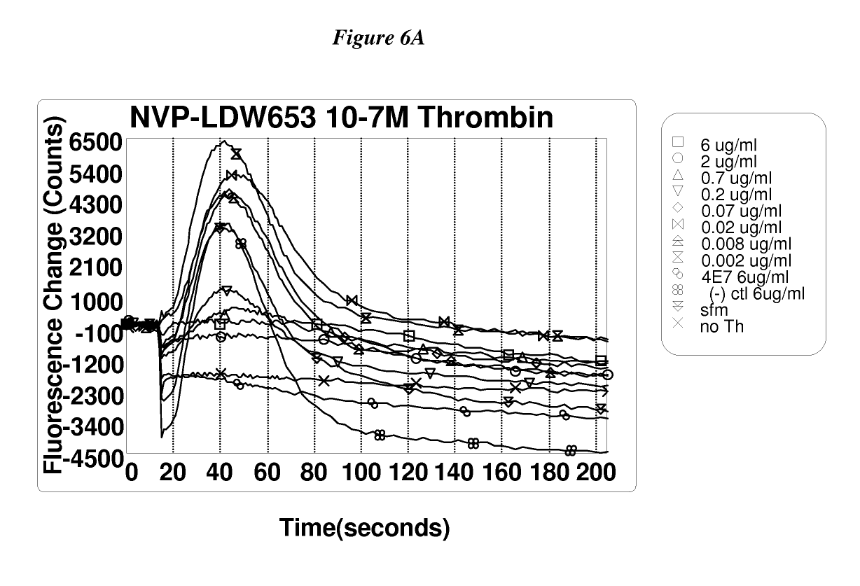

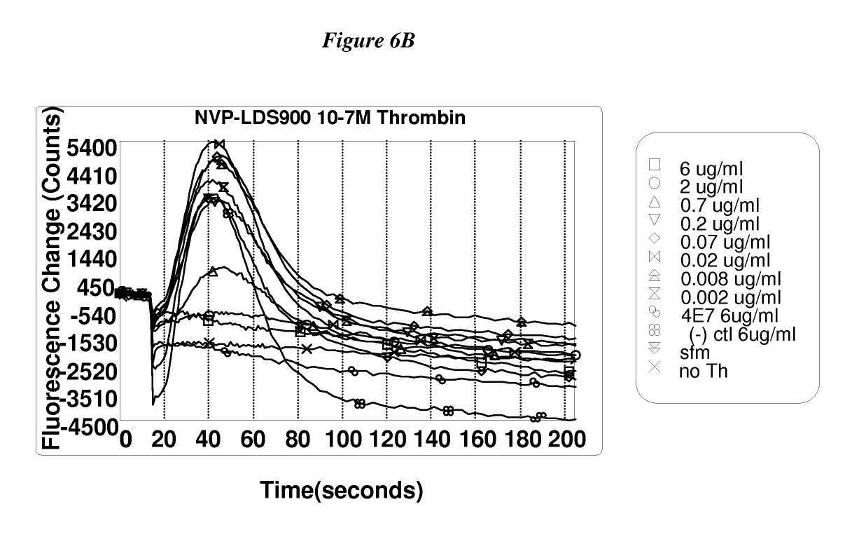

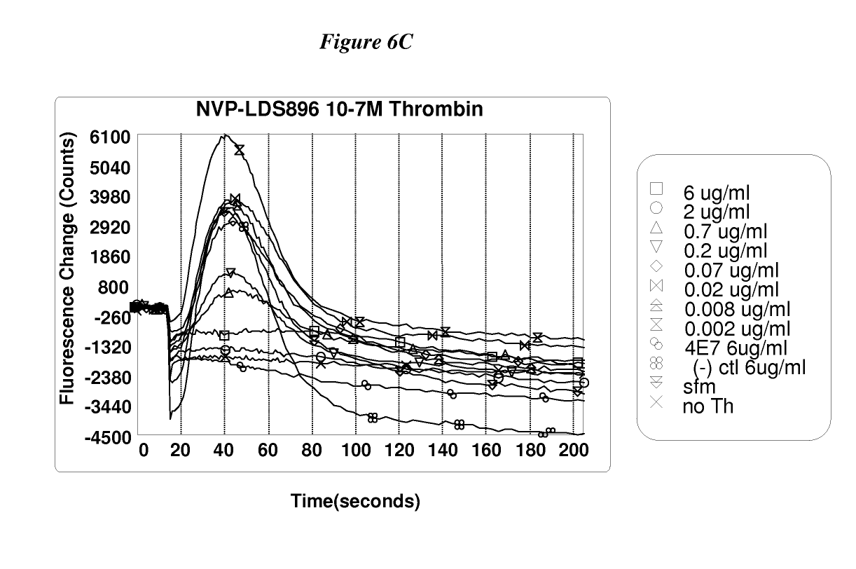

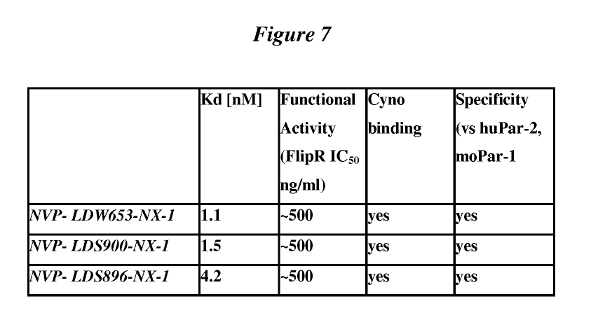

In an alternative approach, a mutagenic library was constructed in which each residue within this CDR2 was mutated to either the murine sequence or the corresponding residue from a single human germline sequence (VH1-46 was the closest human germline sequence to the identified “front-end” cassettes) (see, FIG. 3). This mutagenic library was coupled to selected “front ends” and human framework 3 (FR3) sequences that were seen to support antigen binding. All members of this library had the common CDR3 sequence of the PR15-5 reference Fab′, along with human germ-line FR4 sequences. Thus an engineered human “middle” cassette library was built, screened by colony lift binding assay and antigen binding clones were selected and further analyzed by ELISA and ForteBio Octet. In this way, multiple CDR sequences that support Par-1 antigen binding were identified that have close to human germline amino acid sequence (See FIG. 3).

After the identification of a pool of high affinity Fabs with close to human germline sequence, affinity maturation libraries were built. The common CDR3 sequences of a panel of optimized Fab clones were mutated using degenerate PCR primers to generate libraries. These mutagenic libraries were screened using colony lift binding assay. The selected Fabs were ranked for affinity with ELISA and ForteBio analysis. Mutations that supported equal or improved affinity for antigen when compared to the PR15-5 reference Fab′ were identified. See, Table 2.

### Affinity of Fab′s and Fabs for Human Par-1 Antigen Using Forte Octet Analysis

Fully optimized Fabs isolated from cassette and mutagenic library screens by colony lift binding assay and ELISA were further characterized by kinetic comparison with the reference Fab′ PR15-5. Binding kinetics were analyzed using a ForteBio Octet system for real-time label-free monitoring of protein-protein interactions. Calculated association and dissociation and overall affinity constants are shown in Table 2.

TABLE 2

| | Experiment 1 | Experiment 1 | Experiment 2 | Experiment 2 | Experiment 2 |
|---|---|---|---|---|---|
| | PR15-5 | LDS896 | PR15-5 | LDS900 | LDW653 |
| MolarConc [M] | 1E−8 | 1E−8 | 5E−8 | 2.5E−8 | 2.5E−8 |
| k_{dis} [1/s] | 3.08E−4 | 3.11E−4 | 3.50E−4 | 2.89E−4 | 2.93E−4 |
| k_{assoc} [1/Ms] | 3.95E5 | 2.65E5 | 3.29E5 | 1.96E5 | 2.59E5 |
| K_{D} [M] | 7.79E−10 | 1.18E−9 | 1.06E−9 | 1.47E−9 | 1.13E−9 |

Table 2 shows an analysis of Fab′ and Fab binding to recombinant Par-1 antigen by bio-layer interferometry using ForteBio Octet biosensor technology, showing association rate constant (kₐ), dissociation rate constant (k_{d}) and calculated affinity (K_{D}). Kinetic analysis of the improved Fab clones LDS-896, LDS900 and LDW653 and the reference Fab′ clone PR15-5 demonstrated all to have high affinities for Par-1 antigen.

FIG. 3 shows an amino acid sequence alignment of the V-region sequences of optimized Fabs LDS-896, LDS900 and LDW653 compared with a human germline sequence. The percentage amino acid sequence identities of the reference and optimized V-region sequences to corresponding human germline amino acid sequences are shown in Table 3.

TABLE 3

| | | Vk Identity |
|---|---|---|
| | Vh Identity | to VkII |
| Antibody | to Vh1-46/JH6 | A3/Jk2 |
| 4E7.J14 | 64% | 80% |
| LDS896 | 81% | 92% |
| LDS900 | 88% | 92% |
| LDW653 | 92% | 92% |

All percentage sequence identities in Table 3 represent identity to a single human germline sequence across the V-region and exclude the CDR3 BSD sequences. It can be seen that most of the V-regions of all three improved Fabs are extremely close to a corresponding human germline sequence, with percentage amino acid sequence identities of about 90%.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. An antibody that binds protease activated receptor-1 (PAR1), wherein the antibody comprises
(a) a heavy chain variable region comprising a human heavy chain V-segment, a heavy chain complementary determining region 3 (CDR3), and a heavy chain framework region 4 (FR4), and
(b) a light chain variable region comprising a human light chain V-segment, a light chain CDR3, and a light chain FR4, wherein
i) the heavy chain CDR3 comprises the amino acid sequence DDX₁X₂SX₃WX₄FDV, wherein X₁ is G or I, X₂ is P or Y, X₃ is H, L, P, M, E, W, T, S, Q or A and X₄ is Y or F (SEQ ID NO:10);
ii) the light chain CDR3 variable region comprises the amino acid sequence FQGX₅X₆VPFT, wherein X₅ is S, D, A or V and X₆ is H, R, T, S or K (SEQ ID NO:20);
wherein the antibody is a PAR1 antagonist.

2. The antibody of claim 1, wherein the heavy chain V-segment shares at least 90% sequence identity to SEQ ID NO:41, and wherein the light chain V-segment shares at least 90% sequence identity to SEQ ID NO:46.

3. The antibody of claim 1, wherein the heavy chain V-segment shares at least 90% sequence identity to an amino acid selected from the group consisting of SEQ ID NO:42, SEQ ID NO:43, SEQ ID NO:44 and SEQ ID NO:45, and wherein the light chain V-segment shares at least 90% sequence identity to an amino acid selected from the group consisting of SEQ ID NO:47, SEQ ID NO:48, SEQ ID NO:49 and SEQ ID NO:50.

4. The antibody of claim 1, wherein:
i) the heavy chain CDR3 comprises an amino acid sequence selected from the group consisting of SEQ ID NO:11, SEQ ID NO:12 and SEQ ID NO:13; and
ii) the light chain CDR3 comprises an amino acid sequence selected from the group consisting of SEQ ID NO:22 and SEQ ID NO:23.

5. The antibody of claim 1, wherein the heavy chain FR4 is a human germline FR4.

6. The antibody of claim 5, wherein the heavy chain FR4 is human germline JH6 (WGQGTTVTVSS; SEQ ID NO:32).

7. The antibody of claim 5, wherein the heavy chain J-segment comprises the human germline JH6 partial sequence DVWGQGTTVTVSS (SEQ ID NO:66).

8. The antibody of claim 1, wherein the light chain FR4 is a human germline FR4.

9. The antibody of claim 8, wherein the light chain FR4 is human germline Jk2 (FGQGTKLEIK; SEQ ID NO:40).

10. The antibody of claim 8, wherein the light chain J-segment comprises the human germline Jk2 partial sequence TFGQGTKLEIK (SEQ ID NO:67).

11. The antibody of claim 1, wherein the heavy chain V-segment and the light chain V-segment each comprise a complementary determining region 1 (CDR1) and a complementary determining region 2 (CDR2); wherein:
i) the CDR1 of the heavy chain V-segment comprises an amino acid sequence of SEQ ID NO:2;
ii) the CDR2 of the heavy chain V-segment comprises an amino acid sequence of SEQ ID NO:6;
iii) the CDR1 of the light chain V-segment comprises an amino acid sequence of SEQ ID NO:14; and
iv) the CDR2 of the light chain V-segment comprises an amino acid sequence of SEQ ID NO:17.

12. The antibody of claim 11, wherein
i) the CDR1 of the heavy chain V-segment comprises SEQ ID NO:4;
ii) the CDR2 of the heavy chain V-segment comprises SEQ ID NO:8;
iii) the heavy chain CDR3 comprises SEQ ID NO:11;
iv) the CDR1 of the light chain V-segment comprises SEQ ID NO:16;
v) the CDR2 of the light chain V-segment comprises SEQ ID NO:19; and
vi) the light chain CDR3 comprises SEQ ID NO:22.

13. The antibody of claim 11, wherein
i) the CDR1 of the heavy chain V-segment comprises SEQ ID NO:4;
ii) the CDR2 of the heavy chain V-segment comprises SEQ ID NO:8;
iii) the heavy chain CDR3 comprises SEQ ID NO:12;
iv) the CDR1 of the light chain V-segment comprises SEQ ID NO:16;
v) the CDR2 of the light chain V-segment comprises SEQ ID NO:19; and
vi) the light chain CDR3 comprises SEQ ID NO:23.

14. The antibody of claim 11, wherein
i) the CDR1 of the heavy chain V-segment comprises SEQ ID NO:5;
ii) the CDR2 of the heavy chain V-segment comprises SEQ ID NO:9;
iii) the heavy chain CDR3 comprises SEQ ID NO:13;
iv) the CDR1 of the light chain V-segment comprises SEQ ID NO:16;
v) the CDR2 of the light chain V-segment comprises SEQ ID NO:19; and
vi) the light chain CDR3 comprises SEQ ID NO:23.

15. The antibody of claim 1, wherein the heavy chain variable region shares at least 90% amino acid sequence identity to the variable region of SEQ ID NO:51 and the light chain variable region shares at least 90% amino acid sequence identity to the variable region of SEQ ID NO:55.

16. The antibody of claim 1, wherein the heavy chain variable region shares at least 90% amino acid sequence identity to the variable region selected from the group consisting of SEQ ID NO:52, SEQ ID NO:53 and SEQ ID NO:54 and the light chain variable region shares at least 90% amino acid sequence identity to the variable region selected from the group consisting of SEQ ID NO:57, SEQ ID NO:58 and SEQ ID NO:59.

17. The antibody of claim 1, wherein the heavy chain variable region shares at least 95% amino acid sequence identity to the variable region selected from the group consisting of SEQ ID NO:52, SEQ ID NO:53 and SEQ ID NO:54 and the light chain variable region shares at least 95% amino acid sequence identity to the variable region selected from the group consisting of SEQ ID NO:57, SEQ ID NO:58 and SEQ ID NO:59.

18. The antibody of claim 1, wherein the heavy chain variable region comprises an amino acid sequence selected from the group consisting of SEQ ID NO:52, SEQ ID NO:53 and SEQ ID NO:54 and the light chain variable region comprises an amino acid sequence selected from the group consisting of SEQ ID NO:57, SEQ ID NO:58 and SEQ ID NO:59.

19. The antibody of claim 1, wherein the antibody binds to PAR1 with an equilibrium dissociation constant (K_{D}) of less than 1×10⁻⁸ M.

20. The antibody of claim 1, wherein the antibody is a FAb′ fragment.

21. The antibody of claim 1, wherein the antibody is an IgG.

22. The antibody of claim 1, wherein the antibody is a single chain antibody (scFv).

23. The antibody of claim 1, wherein the antibody comprises human constant regions.

24. The antibody of claim 1, wherein the antibody comprises a heavy chain comprising SEQ ID NO:52 and a light chain comprising SEQ ID NO:57.

25. The antibody of claim 1, wherein the antibody comprises a heavy chain comprising SEQ ID NO:53 and a light chain comprising SEQ ID NO:58.

26. The antibody of claim 1, wherein the antibody comprises a heavy chain comprising SEQ ID NO:54 and a light chain comprising SEQ ID NO:59.

27. A pharmaceutically acceptable composition comprising an antibody of any one of claims 1-26 and a physiologically compatible excipient.

28. A method of ameliorating the symptoms of a disease condition mediated by intracellular signaling through PAR1 comprising administering to a subject in need thereof an antibody of any one of claims 1-26, wherein the antibody is an antagonist of PAR1.

29. The method of claim 28, wherein the disease condition mediated by aberrant intracellular signaling through PAR1 is a chronic intestinal inflammatory disorder.

30. The method of claim 28, wherein the disease condition mediated by aberrant intracellular signaling through PAR1 is a fibrotic disorder.

31. The method of claim 28, wherein the disease condition mediated by aberrant intracellular signaling through PAR1 is a cancer that overexpresses PAR1.

32. The method of claim 28, wherein the disease condition mediated by aberrant intracellular signaling through PAR1 is ischemia-reperfusion injury.

33. An antibody that specifically binds PAR1, wherein the antibody comprises a heavy chain variable region and a light chain variable region, wherein the heavy chain variable region and the light chain variable region each comprise the following three complementary determining regions (CDRs): CDR1, CDR2 and CDR3; wherein:
i) the CDR1 of the heavy chain variable region comprises an amino acid sequence selected from the group consisting of SEQ ID NO:3, SEQ ID NO:4 and SEQ ID NO:5;
ii) the CDR2 of the heavy chain variable region comprises an amino acid sequence selected from the group consisting of SEQ ID NO:7, SEQ ID NO:8 and SEQ ID NO:9;
iii) the CDR3 of the heavy chain variable region comprises an amino acid sequence selected from the group consisting of SEQ ID NO:11, SEQ ID NO:12 and SEQ ID NO:13;
iv) the CDR1 of the light chain variable region comprises an amino acid sequence selected from the group consisting of SEQ ID NO:15 and SEQ ID NO:16;
v) the CDR2 of the light chain variable region comprises an amino acid sequence selected from the group consisting of SEQ ID NO:18 and SEQ ID NO:19;
vi) the CDR3 of the light chain variable region comprises an amino acid sequence selected from the group consisting of SEQ ID NO:22 and SEQ ID NO:23.
